(19) United States
(12) Patent Application Publication
(10) Pub. No.: US 2004/0157932 A1
(43) Pub. Date: Aug. 12, 2004

(54) Supplements and foods comprising oleylethanolamide

(75) Inventor: Asgeir Saebo, Oersta (NO)

Correspondence Address: MEDLEN & CARROLL, LLP, Suite 350, 101 Howard Street, San Francisco, CA, 94105, US

(73) Assignee: Natural ASA,, Lysaker (NO)

(21) Appl. No.: 10/715,705

(22) Filed: Nov. 18, 2003

Related U.S. Application Data
(60) Provisional application No. 60/427,123, filed on Nov. 18, 2002.

(51) Int. Cl. A61K 31/16; A23K 1/165; A23K 1/17

(52) U.S. Cl. 514/625; 424/442

(57) ABSTRACT

The present invention relates to the field of human and animal nutrition, and in particular to food products and dietary and nutritional supplements comprising oleylethanolamide compounds. The food products and supplement of the present invention find use in inducing satiety and decreasing weight in subjects.

This application claims priority from U.S. Provisional Application Serial No. 60/427,123, filed Nov. 18, 2002.

### FIELD OF THE INVENTION

The present invention relates to the field of human and animal nutrition, and in particular to food products and dietary and nutritional supplements comprising oleylethanolamide compounds.

### BACKGROUND OF THE INVENTION

Obesity is a growing public health problem that affects 97 million American adults—55 percent of the population. These individuals are at increased risk of illness from hypertension, lipid disorders, type 2 diabetes, coronary heart disease, stroke, gallbladder disease, osteoarthritis, sleep apnea and respiratory problems, and certain cancers. The total costs attributable to obesity-related disease approaches $100 billion annually.

Obesity occurs when a person's body has too much fat (adipose tissue). Obesity is determined using the body mass index (BMI). The BMI is based on height and weight. People who have BMIs of 19 up to 24.9 are in a healthy weight range. People who have BMIs of 25 up to 29.9 are overweight. People who have BMIs of 30 or higher are obese.

The evidence is solid that the risk for various cardiovascular and other diseases rises significantly when someone's BMI is over 25 and that risk of death increases as the body mass index reaches and surpasses 30. As BMI levels rise, average blood pressure and total cholesterol levels increase and average HDL or good cholesterol levels decrease. Men in the highest obesity category have more than twice the risk of hypertension, high blood cholesterol, or both compared to men of normal weight. Women in the highest obesity category have four times the risk of either or both of these risk factors.

Treatment for obesity is based on diet, exercise, and medications such as appetite suppressants and bulking agents. Many of the currently available treatment methods suffer from side effects and can only be used for a limited period of time. Additional agents that aid in weight loss and maintenance are needed.

### SUMMARY OF THE INVENTION

The present invention relates to the field of human and animal nutrition, and in particular to food products and dietary and nutritional supplements comprising oleylethanolamide compounds. Accordingly, in some embodiments, the present invention provides a composition for a human or animal diet comprising a food product and an oleylethanolamide component. In some embodiments, the composition is for human consumption. In other embodiments, the composition is for non-human animal consumption (e.g., a pet food). In some embodiments, the composition comprises between 0.01 gram and 10.0 grams and preferably between 0.5 grams and 2.0 grams of the oleylethanolamide. In some preferred embodiments, the composition comprises approximately 1.0 gram of the oleylethanolamide. In some embodiments, the oleylethanolamide is a solid fat. In other embodiments, the oleylethanolamide is dissolved in an oil. In still further embodiments, the oleylethanolamide is a powder. In some embodiments, the composition further comprises a conjugated linoleic acid component. In some embodiments, the composition comprises between 0.5 grams and 10.0 grams of the conjugated linoleic acid component. In some preferred embodiments, the composition comprises approximately 3.6 grams of the conjugated linoleic acid component. In some embodiments, the conjugated linoleic acid is an oil. In other embodiments, the conjugated linoleic acid is a powder. The present invention is not limited to a particular food product. In some embodiments, the food product is selected from the group including, but not limited to, a beverage, a prepared food, a food ingredient, and a dairy product. In further preferred embodiments, the composition further comprises a fatty acid amide hydrolase inhibitor. The present invention is not limited to a particular fatty acid hydrolase inhibitor. In further embodiments, the oleylethanolamide component is a derivative of OEA. The present invention is not limited to a particular kind of OEA derivative. In some embodiments, the OEA derivative is the reaction product of OEA and a primary amine. In other embodiments, the OEA derivative is the Ca salt of OEA.

The present invention further provides a composition comprising a dietary supplement, the dietary supplement comprising oleylethanolamide. In some embodiments, the dietary supplement comprises between 0.01 gram and 10.0 grams, and preferably between 0.5 and 10.0 grams of the oleylethanolamide. In some preferred embodiments, the dietary supplement comprises approximately 1.0 gram of the oleylethanolamide. In some embodiments, the oleylethanolamide is a solid fat. In other embodiments, the oleylethanolamide is a powder. In some embodiments, the dietary supplement further comprises a conjugated linoleic acid component. In some embodiments, the dietary supplement comprises between 0.5 grams and 10.0 grams of the conjugated linoleic acid component. In some preferred embodiments, the dietary supplement comprises approximately 3.6 grams of the conjugated linoleic acid component. In some embodiments, the conjugated linoleic acid is an oil. In other embodiments, the conjugated linoleic acid is a powder. In some embodiments, the dietary supplement is in a capsule. In some embodiments, the capsule has an enteric coating. In some embodiments, the capsule is a gelatin capsule. In further preferred embodiments, the composition further comprises a fatty acid amide hydrolase inhibitor. The present invention is not limited to a particular fatty acid hydrolase inhibitor. In further embodiments, the composition comprises a derivative of OEA. The present invention is not limited to a particular kind of OEA derivative. In some embodiments, the OEA derivative is the reaction product of OEA and a primary amine. In other embodiments, the OEA derivative is the Ca salt of OEA.

The present invention additionally provides a nutritional supplement, the nutritional supplement comprising oleylethanolamide. In some embodiments, the nutritional supplement comprises between 0.01 gram and 10.0 grams and preferably between 0.5 grams and 10.0 grams of the oleylethanolamide. In some embodiments, the nutritional supplement comprises approximately 1.0 gram of the oleylethanolamide. In some embodiments, the oleylethanolamide is a solid fat. In other embodiments, the oleylethanolamide is a powder. In some embodiments, the nutritional supplement further comprises a conjugated linoleic acid component. In some embodiments, the composition comprises between 0.5 grams and 10.0 grams of the conjugated linoleic acid component. In some preferred embodiments, the composition comprises approximately 3.6 grams of the conjugated linoleic acid component. In some embodiments, the conjugated linoleic acid is an oil. In other embodiments, the conjugated linoleic acid is a powder. The present invention is not limited to a particular nutritional supplement. A variety of nutritional supplements are contemplated, including, but not limited to, nutrient bars, nutrient beverages or nutrient beverage concentrates. In further preferred embodiments, the composition further comprises a fatty acid amide hydrolase inhibitor. The present invention is not limited to a particular fatty acid hydrolase inhibitor. In further embodiments, the composition comprises a derivative of OEA. The present invention is not limited to a particular kind of OEA derivative. In some embodiments, the OEA derivative is the reaction product of OEA and a primary amine. In other embodiments, the OEA derivative is the Ca salt of OEA.

In still further embodiments, the present invention provides a method of supplementing a diet, comprising orally administering a composition comprising oleylethanolamide. In some embodiments, the composition comprises between 0.01 gram and 10.0 grams and preferably between 0.5 grams and 2.0 grams of the oleylethanolamide. In some preferred embodiments, the composition comprises approximately 1.0 gram of the oleylethanolamide. In some embodiments, the oleylethanolamide is a solid fat. In other embodiments, the oleylethanolamide is dissolved in an oil. In still further embodiments, the oleylethanolamide is a powder. In some embodiments, the composition further comprises a conjugated linoleic acid component. In some embodiments, the composition comprises between 0.5 grams and 10.0 grams of the conjugated linoleic acid component. In some preferred embodiments, the composition comprises approximately 3.6 grams of the conjugated linoleic acid component. In some embodiments, the conjugated linoleic acid is an oil. In other embodiments, the conjugated linoleic acid is a powder. In some embodiments, the composition is a dietary supplement. In some embodiments, the dietary supplement is a capsule. In other embodiments, the composition is a nutritional supplement. In some embodiments, the nutritional supplement is selected from the group including, but not limited to, a nutrient bar, a nutrient beverage and a nutrient beverage concentrate. In still further embodiments, the composition is a food product (e.g., including, but not limited to, a beverage, a prepared food product, a food ingredient, and a dairy product). In yet other embodiments, the composition is a foodstuff. In further preferred embodiments, the composition further comprises a fatty acid amide hydrolase inhibitor. The present invention is not limited to a particular fatty acid hydrolase inhibitor. In further embodiments, the composition comprises a derivative of OEA. The present invention is not limited to a particular kind of OEA derivative. In some embodiments, the OEA derivative is the reaction product of OEA and a primary amine. In other embodiments, the OEA derivative is the Ca salt of OEA.

In yet other embodiments, the present invention provides a method, comprising providing a oleic acid composition; and treating the oleic acid composition under conditions such that an oleylethanolamide composition is generated, the oleylethanolamide composition suitable for consumption by a human or animal. In some embodiments, the under conditions such that comprise reacting the oleic acid composition with a chloride to produce the acid chloride of oleic acid, reacting the acid chloride with ethanolamideaceteate to generate an acetate of oleylethanoloamide and hydrolyzing the acetate to generate oleylethanolamide. In some embodiments, the method further comprises the step of combining the oleylethanolamide with a food product. In some embodiments, the food product is a pet food. In some embodiments, the food product is combined with between 0.01 gram and 10.0 grams and preferably between 0.5 grams and 2.0 grams of the oleylethanolamide. In some preferred embodiments, the food product is combined with approximately 1.0 gram of the oleylethanolamide. In further preferred embodiments, the composition further comprises a fatty acid amide hydrolase inhibitor. The present invention is not limited to a particular fatty acid hydrolase inhibitor. In further embodiments, the composition comprises a derivative of OEA. The present invention is not limited to a particular kind of OEA derivative. In some embodiments, the OEA derivative is the reaction product of OEA and a primary amine. In other embodiments, the OEA derivative is the Ca salt of OEA.

In still additional embodiments, the present invention provides a composition for a human or animal diet comprising a food product and a fatty acid amide component. In some embodiments, the composition is for human consumption. In other embodiments, the composition is for non-human animal consumption (e.g., a pet food). In some embodiments, the composition comprises between 0.01 gram and 10.0 grams and preferably between 0.5 grams and 2.0 grams of the fatty acid amide. In some preferred embodiments, the composition comprises approximately 1.0 gram of the fatty acid amide. In some embodiments, the fatty acid amide is a solid fat. In other embodiments, the fatty acid amide is dissolved in an oil. In yet other embodiments, the fatty acid amide is a powder. The present invention is not limited to a particular food product. Any number of food products are contemplated including, but not limited to, a beverage, a prepared food, a food ingredient, and a dairy product. In some embodiments, the fatty acid is an essential fatty acid. In some embodiments, the fatty acid is a C: 18 or longer fatty acid. In some embodiments, the fatty acid is an unsaturated fatty acid. In other embodiments, the fatty acid is a saturated fatty acid. In some embodiments, the fatty acid is a conjugated fatty acid. In some embodiments, the fatty acid is selected from the group including, but not limited to, conjugated linoleic acid, oleic acid, docosahexaenoic acid, gamma-linolenic acid, alpha linoleic acid, stearidonic acid, dihomo-gamma-linolenic acid, arachidonic acid, eicosapentaenoic acid, adrenic acid, docosahexaenoic acid, and docosapentaenoic acid. In further preferred embodiments, the composition further comprises a fatty acid amide hydrolase inhibitor. The present invention is not limited to a particular fatty acid hydrolase inhibitor. In further embodiments, the composition comprises a derivative of OEA. The present invention is not limited to a particular kind of OEA derivative. In some embodiments, the OEA derivative is the reaction product of OEA and a primary amine. In other embodiments, the OEA derivative is the Ca salt of OEA.

Definitions

As used herein, the terms “fatty acid ethanolamide” and “FAE” refers to a fatty acid comprising a long carbon chain (e.g., a saturated, mono-unsaturated, or poly-unsaturated carbon chain) linked to an ethanolamide. Exemplary FAEs are shown in FIG. 1 and include, but are not limited to, oleyl ethanolamide (OEA), palmitylethanolamide (PEA), and arachidylethanolamide (anandamide).

As used herein, the terms “oleylethanolamide” and “OEA” refer to a fatty acid ethanolamide comprising an oleyl carbon chain. It is intended that this term encompass and indicate all positional and geometric isomers of oleylethanolamide with one conjugated carbon-carbon double bond any place in the molecule.

As used herein, the term “oil” refers to a free flowing liquid containing long chain fatty acids (e.g., OEA, CLA), triglycerides, or other long chain hydrocarbon groups. The long chain fatty acids, include, but are not limited to the various isomers of OEA or CLA.

As used herein, the term “physiologically acceptable carrier” refers to any carrier or excipient commonly used with oily pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “dietary supplement” refers to a small amount of a compound for supplementation of a human or animal diet (e.g., OEA) packaged in single or multiple does units. Dietary supplements do not generally provide significant amounts of calories but may contain other micronutrients (e.g., vitamins or minerals). In preferred embodiments, the compound for supplementation of the diet (e.g., OEA) provides a health benefit to the human or animal ingesting the compound.

As used herein, the term “nutritional supplement” refers to a composition comprising a “dietary supplement” in combination with a source of calories. In some embodiments, nutritional supplements are meal replacements or supplements (e.g., nutrient or energy bars or nutrient beverages or concentrates).

As used herein, “conjugated linoleic acid” or “CLA” refers to any conjugated linoleic acid or octadecadienoic free fatty acid. It is intended that this term encompass and indicate all positional and geometric isomers of linoleic acid with two conjugated carbon-carbon double bonds any place in the molecule. CLA differs from ordinary linoleic acid in that ordinary linoleic acid has double bonds at carbon atoms 9 and 12. Examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid and 10,12 octadecadienoic acid, 11, 13 octadecadienoic acid. As used herein, “CLA” encompasses a single isomer, a selected mixture of two or more isomers, and a non-selected mixture of isomers obtained from natural sources, as well as synthetic and semisynthetic CLA.

### DETAILED DESCRIPTION OF THE INVENTION

The present invention relates to the field of human and animal nutrition, and in particular to food products and dietary and nutritional supplements comprising oleylethanolamide compounds. Accordingly, in some embodiments, the present invention provides methods of producing OEA for use in food products and supplements, as well as dietary supplements, nutritional supplements, and food products comprising OEA. In some further embodiments, the supplements and foods additionally comprise CLA.

I. OEA

Oleylethanolamide (OEA) is a natural analogue of the endogenous cannabinoid anandamide. Like anandamide, OEA is produced in cells in a stimulus-dependent manner and is rapidly eliminated by enzymatic hydrolysis, suggesting a function in cellular signaling. However, OEA does not activate cannabinoid receptors and its biological functions are still unknown.

Fatty acid ethanolamides (FAEs) are unusual components of animal and plant lipids (Bachur et al., J. Biol. Chem. 240:1019 [1965]; Chapman et al., Chem. Phys. Lipids 108:221 [2000]) that are synthesized in response to variety of physiological and pathological stimuli, including activation of neurotransmitter receptors in rat brain neurons (Guiffrida et al., Nature Neurosci. 2:358 [1999]) and exposure to metabolic stressors in mouse epidermal cells (Berdyshev et al., Biochem J. 346:369). The primary mechanism underlying FAE generation in mammalian tissues involves two concerted biochemical reactions: cleavage of the membrane phospholipid N-acyl phosphatidylethanolamine (NAPE), catalysed by an unknown phospholipase D; and NAPE re-synthesis, mediated by an N-acyltransferase (NAT) that is regulated by calcium ions and cyclic AMP. After release, FAEs are transported back into cells and eventually broken down to fatty acid and ethanolamine by an intracellular fatty acid amide hydrolase (FAAH). That animal cells release FAEs in a stimulus-dependent manner suggests that these compounds may participate in cell-to-cell communication. Further support for this idea comes from the discovery that the polyunsaturated FAE anandamide (arachidony lethanolatnide) serves as an endogenous ligand for cannabinoid receptors (Devane et al., Science 258:1946 [1992]). However, the pharmacological effects of saturated or mono-unsaturated FAEs such as OEA cannot be accounted for by activation of any of the known cannabinoid receptor subtypes, and the biological roles of these compounds remain elusive.

Rats given OEA in their diets exhibited decreased weight gain and reduced desire for food (Fonseca et al., Nature 414:209 [2001]). In contrast to currently available weight loss drugs, OEA provided the added benefit of not effecting the nervous system of the rats. The present invention is not limited to a particular mechanism. Indeed, an understanding of the mechanism is not necessary to practice the present invention. Nonetheless, it is contemplated that OEA provides an efficient agent for decreasing weight and increasing satiety without the side effects of weight loss drugs. Accordingly, in some embodiments, the present invention provides OEA compounds for use in supplements and in food products.

The present invention further provides coadministration of OEA with a fatty acid amide hydroloase (FAAH) inhibitor. FAAH is an integral membrane protein that degrades fatty acid primary amides and ethanolamides, including OEA (see, e.g., M. P. Patricelli, et al., (1998) Biochemistry 37, 15177-15187; D. G. Deutsch, et al., (1993) Biochem. Pharmacol. 46, 791-796; F. Desamaud, et al., (1995) J. Biol. Chem. 270, 6030-6035; C. J. Hillard, et al., (1995) Biochim. Biophys. Acta 1257, 249-256; N. Ueda, et al., (1995) J. Biol. Chem. 270, 23823-23827; R. L. Omeir, et al., (1995) Life Sci. 56, 1999-2005; S. Maurelli, et al., (1995) FEBS Lett. 377, 82-86; and M. Maccarrone, et al., (1998). J. Biol. Chem. 273, 32332-32339; each herein incorporated by reference in their entirety). The distribution of FAAH in the CNS suggests that it degrades neuromodulating fatty acid amides at their sites of action and is intimately involved in their regulation (see, e.g., E. A. Thomas, et al., (1997) J. Neurosci. Res. 50, 1047-1052; herein incorporated by reference in its entirety). FAAH hydrolyzes a wide range of oleyl and arachidonyl amides, the CB 1 agonist 2-arachidonylglycerol, the related 1-arachidonylglycerol and 1-oleylglycerol, and methyl arachidonate, illustrating a range of bioactive fatty acid amide or ester substrates. (see, e.g., W. Lang, et al., (1999) J. Med. Chem. 42, 896-902; S. K. Goparaju, et al., (1998) FEBS Lett. 442, 69-73; Y. Kurahashi, et al., (1997) Biochem. Biophys. Res. Commun. 237, 512-515; and T. Bisogno, et al., (1997) Biochem. J. 322, 671. Di Marzo, V., T. Bisogno, et al., (1998) Biochem. J. 331, 15-19; each herein incorporated by reference in their entirety). Although a range of fatty acid primary amides are hydrolyzed by the enzyme, FAAH appears to work most effectively on arachidonyl and oleyl substrates (see, e.g., B. F. Cravatt, et al., (1996) Nature 384, 83-87; and D. K. Giang, et al., (1997) Proc. Natl. Acad. Sci. USA 94, 2238-2242; each herein incorporated by reference in their entirety).

Inhibition of FAAH prevents the degradation of endocannabinoids (i.e. anandamides, OEA). In some embodiments of the present invention, FAAH inhibitors are coadministered with OEA in order to prevent the degradation of OEA by FAAH. The present invention is not limited to any particular type of FAAH inhibitor. Several FAAH inhibitors are available. Amongst these is the potent endogenous inhibitor 2-octyl y-gamma.-bromoacetoacetate, which was discovered prior to FAAH and characterized as an endogenous sleep-inducing compound (see, M. P. Patricelli, et al., (1998) Bioorg. Med. Chem. Lett. 8, 613-618; and S. Torii, et al., (1973) Psychopharmacologia 29, 65-75; each herein incorporated by reference in their entirety). Additional inhibitors employ a fatty acid structure attached to pharmacophoric head group. The pharmacophoric head groups can generally be classified as either reversible or irreversible. Reversible inhibitors include electrophilic carbonyl moieties (e.g., trifluoromethyl ketones, α-halo ketones, α-keto esters and amides, and aldehydes). Irreversible inhibitors include sulfonyl fluorides and fluorophosphonates. (see, e.g., B. Koutek, et al., (1994) J. Biol. Chem. 269, 22937-22940; J. E. Patterson, et al., (1996) J. Am. Chem. Soc. 118, 5938-5945; D. L. Boger, et al., (1999) Bioorg. Med. Chem. Lett. 9, 167-172; D. G. Deutsch, et al., (1997) Biochem. Pharmacol. 53, 255-260. D. G. Deutsch, et al., (1997) Biochem. Biophys. Res. Commun. 231, 217-221; and L. De Petrocellis, et al., (1997) Biochem. Biophys. Res. Commun. 231, 82-88; and L. De Petrocellis, et al., (1998) In Recent Advances Prostaglandin, Thromboxane, and Leukotriene Research, Plenum Press: New York, 259-263; each herein incorporated by reference in their entirety). In addition, numerous FAAH inhibitors are throroughly described in U.S. Pat. Nos. 6,462,054 and 6,562,846 (each herein incorporated by reference in their entirety).

The present invention further provides coadministration of OEA with agents inhibiting unsaturated fatty acid biohydrogenation. Biohydrogenation refers to the microbial process of saturating a compound with hydrogen, which would convert unsaturated fatty acids into saturated free fatty acids. Unsaturated fatty acids typically undergo biohydrogenation by bacteria that results in the production of high levels of saturated free fatty acids. As such, normally when subjects (e.g., humans, cows, pigs, rats) ingest unsaturated fatty acids, they are converted to saturated fatty acids and absorbed into the tissues of the subject (see, e.g., U.S. Pat. No. 5,547,686; herein incorporated in its entirety).

In some embodiments of the present invention, agents that inhibit fatty acid biohydrogenation are coadministered with OEA in order to increase OEA absorption. The present invention is not limited to any particular type of agent that inhibits fatty acid biohydrogenation. Several agents that inhibit fatty acid biohydrogenation are available. Amongst these include, for example, the product of the reaction of OEA with primary amines (see, e.g., U.S. Pat. No. 5,547,686; herein incorporated in its entirety) and Ca salts of OEA (see, e.g., Perfield, et al (2002) J. Dairy Science, 85, 2609-2617; Bernal-Santos et al (2001) J. Dairy Science, 84(Supp. 1), 82 (Abstract); Giesy et al (1999) J. Dairy Science, 82(Supp. 1); 83 (Abstract); Medeiros et al (2000) J. Dairy Science, 83(Supp. 1); 169 (Abstract); each of which is herein incorporated in its entirety).

II. Synthesis of OEA Containing Compounds

OEA and other FAE compounds may be synthesized using any suitable method. For example, in some embodiments, the method described in Giuffrida et al. (Giuffrida and Piomelli, in Lipid Second Messengers Eds. Laychock, S. G. and Rubin R. P. 113-133 CRC, Boca Raton, 1998) and Giuffrida, A. et al. (1998) FEBS Lett., 422, 373-376) is utilized.

In other embodiments, the present invention provides an inexpensive method of producing OEA from natural oils (See e.g., Example 1). Preferred oils are those that have a high content of oleic acid and a low content of linoleic acid. Exemplary oils include, but are not limited to, olive oil and oleic acid.

In some embodiments, palmitic acid (C 16:0) is first removed by fractionation from the free fatty acid preparation of the selected oil. In some embodiments, residues of palmitic acid and stearic acid are next removed by urea fractionation (e.g., using urea complexation techniques). The resulting fatty acid mixture then comprises primarily oleic acid (approximately 95%) and linoleic acid (approximately 5%). From this mixture acid chlorides are prepared and then reacted with an ester of ethanolamide (with a protected OH group). The ester is then cleaved to yield the free hydroxy group on the finished OEA. The final mixture contains a small amount of lioelylethanolamide (approximately 5%).

III. Delivery of OEA Containing Compounds

The OEA containing compounds of the present invention may be delivered in any suitable format including, but not limited to, those described herein. In preferred embodiments, OEA is formulated for delivery in the intestines. The present invention is not limited to a particular mechanism. Indeed, an understanding of the mechanism is not necessary to practice the present invention. Nonetheless, it is contemplated that cell in the intestinal mucosa are the target cell for OEA. It is further contemplated that release of the OEA close to the target cells will reduce loss of the active ingredient due to hydrolysis of OEA by FAAH to free oleic acid and ethanolamide.

A. Dietary Supplements

In some embodiments, the present invention provides dietary supplements comprising OEA. In some embodiments, the OEA for use in a dietary supplement is an oil. However, in preferred embodiments, the OEA is a powder. The ingredients of the dietary supplement of this invention are contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington 's Pharmaceutical Sciences (Maack Publishing Co., Easton, Pa.).

In other embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: asorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

Dietary supplements may contain between 0.1 g and 10.0 g of OEA, preferably between 0.5 g and 2.0 g OEA, and even more preferably, approximately 1.0 g OEA. In some embodiments, the dietary supplement further comprises conjugated linoleic acid (CLA; See e.g., below description of CLA). In such embodiments, the dietary supplement may comprise between 0.1 g and 10.0 g of CLA, preferably between 0.5 g and 2.0 g CLA, and even more preferably, approximately 1.0 g CLA.

The dietary supplements of the present invention may be taken one or more times daily. Preferably, the dietary supplement is administered orally one to two times daily. Frequency of administration will, of course, depend on the dose per unit (capsule or tablet) and the desired level of ingestion. Dose levels/unit can be adjusted to provide the recommended levels of ingredients per day (e.g., approximately 1 g of OEA) in a reasonable number of units (e.g., two capsules or tablets taken twice a day). In preferred embodiments, the doses add up each day to the daily intake of each ingredient. In preferred embodiments, the dietary supplements are taken with meals or before meals. In other embodiments, the dietary supplements are not taken with meals. In preferred embodiments, the OEA increases satiety and results in a decrease in caloric intake and subsequent weight loss.

B. Nutritional Supplements

In other embodiments, the present invention provides nutritional supplements (e.g., energy bars or meal replacement bars or beverages) comprising OEA. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

Servings of the nutritional supplement preferably contain between 0.1 g and 10.0 g of OEA, preferably between 0.5 and 2.0 g OEA, and even more preferably approximately 1.0 g OEA. In some embodiments, the nutritional supplements of the present invention comprise further comprise CLA (e.g., in the amounts described above for dietary supplements). A nutritional supplement, which supplies, in a recommended daily intake, nutrients comprising those listed above and OEA (and optionally CLA), can be ingested in various amounts throughout a given day. It is understood by those of skill in the art that other ingredients can be added to those described herein, for example, fillers, emulsifiers, preservatives, etc. for the processing or manufacture of a nutritional supplement.

C. Food Products

In still further embodiments, the present invention provides food products, prepared food products, or foodstuffs comprising OEA. For example, in some embodiments, beverages and solid or semi-solid foods comprising OEA are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

Servings of the food product preferably contain between 0.1 g and 10.0 g of OEA, preferably between 0.5 and 2.0 g OEA, and even more preferably approximately 1.0 g OEA. In some embodiments, the food products further comprise CLA (e.g., in the amounts described above for dietary supplements).

D. CLA

In some embodiments, the dietary supplements, nutritional supplements, food products, prepared food products, and food stuffs of the present invention further comprise conjugated linoleic Acid (CLA) compounds (See e.g., U.S. Pat. Nos. 6,410,761; 6,333,353; 6,242,621; 6,225,486; 6,015,833; each of which is herein incorporated by reference). In 1978, researchers at the University of Wisconsin discovered the identity of a substance contained in cooked beef that appeared to inhibit mutagenesis. The substance was found to be a mixture of positional isomers of linoleic acid (C18:2) having conjugated double bonds. The c9, t11 and t10, c12 isomers are present in greatest abundance, but it is uncertain which isomers are responsible for the biological activity observed. It has been noted from labeled uptake studies that the 9,11 isomer appears to be somewhat preferentially taken up and incorporated into the phospholipid fraction of animal tissues, and to a lesser extent the 10,12 isomer. (Ha, et al., Cancer Res., 50: 1097 [1990]).

The biological activity associated with conjugated linoleic acids (termed CLA) is diverse and complex. At present, very little is known about the mechanisms of action, although several preclinical and clinical studies in progress are likely to shed new light on the physiological and biochemical modes of action. The anticarcinogenic properties of CLA have been well documented. Administration of CLA inhibits rat mammary tumorigenesis, as demonstrated by Birt, et al., Cancer Res., 52: 2035s [1992]. Ha, et al., Cancer Res., 50: 1097 [1990] reported similar results in a mouse forestomach neoplasia model. CLA has also been identified as a strong cytotoxic agent against target human melanoma, colorectal and breast cancer cells in vitro. Review articles confirm the conclusions drawn from individual studies (See e.g., Ip, Am. J. Clin. Nutr., 66 (6 Supp): 1523s [1997]).

Although the mechanisms of CLA action are still obscure, there is evidence that some component(s) of the immune system may be involved, at least in vivo. U.S. Pat. No. 5,585,400 (Cook, et al., incorporated herein by reference), discloses a method for attenuating allergic reactions in animals mediated by type I or TgE hypersensitivity by administering a diet containing CLA. CLA in concentrations of about 0.1 to 1.0 percent was also shown to be an effective adjuvant in preserving white blood cells. U.S. Pat. No. 5,674,901 (Cook, et al.), incorporated herein by reference, disclosed that oral or parenteral administration of CLA in either free acid or salt form resulted in elevation in CD-4 and CD-8 lymphocyte subpopulations associated with cell-mediated immunity. Adverse effects arising from pretreatment with exogenous tumor necrosis factor could be alleviated indirectly by elevation or maintenance of levels of CD-4 and CD-8 cells in animals to which CLA was administered. Finally, U.S. Pat. No. 5,430,066, incorporated herein by reference, describes the effect of CLA in preventing weight loss and anorexia by immune stimulation.

Apart from potential therapeutic and pharmacologic applications of CLA as set forth above, there has been much excitement regarding the use of CLA nutritively as a dietary supplement. CLA has been found to exert a profound generalized effect on body composition, in particular redirecting the partitioning of fat and lean tissue mass. U.S. Pat. No. 5,554,646 (Cook, et al.), incorporated herein by reference, discloses a method utilizing CLA as a dietary supplement in which pigs, mice, and humans were fed diets containing 0.5 percent CLA. In each species, a significant drop in fat content was observed with a concomitant increase in protein mass. It is interesting that in these animals, increasing the fatty acid content of the diet by addition of CLA resulted in no increase in body weight, but was associated with a redistribution of fat and lean within the body. Another dietary phenomenon of interest is the effect of CLA supplementation on feed conversion. U.S. Pat. No. 5,428,072 (Cook, et al., incorporated herein by reference), provided data showing that incorporation of CLA into animal feed (birds and mammals) increased the efficiency of feed conversion leading to greater weight gain in the CLA supplemented animals. The potential beneficial effects of CLA supplementation for food animal growers is apparent.

Another important source of interest in CLA, and one which underscores its early commercial potential, is that it is naturally occurring in foods and feeds consumed by humans and animals alike. In particular, CLA is abundant in products from ruminants. For example, several studies have been conducted in which CLA has been surveyed in various dairy products. Aneja, et al., J. Dairy Sci., 43: 231 [1990] observed that processing of milk into yogurt resulted in a concentration of CLA. (Shanta, et al., Food Chem., 47: 257 [1993]) showed that a combined increase in processing temperature and addition of whey increased CLA concentration during preparation of processed cheese. In a separate study, Shanta, et al., J. Food Sci., 60: 695 [1995] reported that while processing and storage conditions did not appreciably reduce CLA concentrations, they did not observe any increases. In fact, several studies have indicated that seasonal or interanimal variation can account for as much as three fold differences in CLA content of cows milk. (See e.g., Parodi, et al., J. Dairy Sci., 60: 1550 [1977]). Also, dietary factors have been implicated in CLA content variation, as noted by Chin, et al., J. Food Camp. Anal., 5: 185 [1992]. Because of this variation in CLA content in natural sources, ingestion of prescribed amounts of various foods will not guarantee that the individual or animal will receive the optimum doses to ensure achieving the desired nutritive effect. Thus, in preferred embodiments, CLA used in foods and supplements of the present invention comprises purified CLA.

Linoleic acid is an important component of biolipids, and comprises a significant proportion of triglycerides and phospholipids. Linoleic acid is known as an “essential” fatty acid, meaning that the animal must obtain it from exogenous dietary sources since it cannot be autosynthesized. Incorporation of the CLA form of linoleic acid may result in a direct substitution of CLA into lipid positions where unconjugated linoleic would have migrated. However, this has not been proven, and some of the highly beneficial but unexplained effects observed may even result from a repositioning of CLA within the lipid architecture at sites where unconjugated linoleic acid would not have otherwise migrated. It is now clear that one source of animal CLA, especially in dairy products, comes from the biochemical action of certain rumen bacteria on native linoleic acid, first isomerizing the linoleic acid to CLA, and then secreting it into the rumen cavity. Kepler, et al., J. Nutrition, 56: 1191 [1966] isolated a rumen bacterium, Butyrivibrio fibrisolvens, which catalyzes formation of 9,11-CLA as an intermediate in the biohydrogenation of linoleic acid. Chin, et al., J. Nutrition, 124: 694 [1994] further found that CLA found in the tissues of rodents was associated with bacteria, since corresponding germ-free rats produced no CLA.

The present invention is not limited to a particular mechanism. Indeed, an understanding of the mechanism is not necessary to practice the present invention. Nonetheless, it is contemplated that a beneficial synergy is obtained from simultaneous reduction of caloric intake from OEA administration and reduction of fat synthesis from CLA. Accordingly, in the some embodiments, the present invention provides nutritional supplements, dietary supplements, food products, and food stuffs comprising a combination of CLA and OEA. In some preferred embodiments, the CLA utilized is a powder form of CLA (See e.g., U.S. Patent Application US20020013365A1; herein incorporated by reference).

E. Additional Fatty Acids

The present invention is not limited to the use of OEA. Indeed, any number of fatty acid amides are contemplated to be useful for inclusion in the food products and nutritional supplements of the present invention. Particularly preferred fatty acid amides are those of essential fatty acids (e.g., C:18 or longer). The present invention contemplated fatty acid amides of saturated, unsaturated and conjugated fatty acids. Exemplary fatty acids amides include, but are not limited to, CLA, oleic acid, docosahexaenoic acid, gamma-linolenic acid, alpha linoleic acid, stearidonic acid, dihomo-gamma-linolenic acid, arachidonic acid, eicosapentaenoic acid, adrenic acid, docosahexaenoic acid, and docosapentaenoic acid.

Fatty acid amides may be synthesized from the free fatty acid using any suitable method including, but not limited to, those disclosed herein.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); M (micromolar); kg (kilograms); g (grams); mg (milligrams); g (micrograms); ng (nanograms); L or l (liters); ml (milliliters); l (microliters); cm (centimeters); mm (millimeters); nm (nanometers); ° C. (degrees centigrade); KOH (potassium hydroxide); HCL (hydrochloric acid); Hg (mercury).

### Example 1

Preparation of OEA

OEA is prepared from fully refined olive oil. The free fatty acids are first prepared by hydrolysis. The saturated fats are then removed. The resulting fractionated concentrate obtained contains primarily oleic acid and is free of sterols and other unsaponifiable matter found in olive oil. The concentrate is then reacted with a chloride to produce the acid chloride of oleic acid. The acid chlorides are then reacted with ethanolamideaceteate. Finally, the ester is hydrolyzed to remove acetic acid under vacuum.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A composition for a human or animal diet comprising a food product and an oleylethanolamide component.

2. The composition of claim 1, wherein said composition is for human consumption.

3. The composition of claim 1, wherein said food product is a pet food.

4. The composition of claim 1, wherein said composition comprises between 0.01 gram and 10.0 grams of said oleylethanolamide.

5. The composition of claim 1, wherein said composition comprises between 0.5 grams and 2.0 grams of said oleylethanolamide.

6. The composition of claim 1, wherein said composition comprises approximately 1.0 gram of said oleylethanolamide.

7. The composition of claim 1, further comprising a conjugated linoleic acid component.

8. The composition of claim 1, wherein said food product is selected from the group consisting of a beverage, a prepared food, a food ingredient, and a dairy product.

9. The composition of claim 1, further comprising a fatty acid amide hydrolase inhibitor.

10. The composition of claim 1, wherein said oleylethanolamide component is a derivative of OEA.

11. A composition comprising a dietary supplement, said dietary supplement comprising oleylethanolamide.

12. The composition of claim 11, wherein said dietary supplement comprises between 0.01 gram and 10.0 grams of said oleylethanolamide.

13. The composition of claim 11, wherein said dietary supplement comprises between 0.5 grams and 10.0 grams of said oleylethanolamide.

14. The composition of claim 11, wherein said dietary supplement comprises approximately 1.0 gram of said oleylethanolamide.

15. The composition of claim 11, wherein said dietary supplement further comprises a conjugated linoleic acid component.

16. The composition of claim 1 1, wherein said dietary supplement is in a capsule.

17. The composition of claim 16, wherein said capsule has an enteric coating.

18. The composition of claim 16, wherein said capsule is a gelatin capsule.

19. The composition of claim 11, further comprising a fatty acid amide hydrolase inhibitor.

20. The composition of claim 11, wherein said composition comprises a derivative of OEA.

21. A method of supplementing a diet, comprising orally administering a composition comprising oleylethanolamide.

22. The method of claim 21, wherein said composition comprises between 0.01 gram and 10.0 grams of said oleylethanolamide.

23. The method of claim 21, wherein said composition comprises between 0.5 grams and 10.0 grams of said oleylethanolamide.

24. The method of claim 21, wherein said composition comprises approximately 1.0 gram of said oleylethanolamide.

25. The method of claim 21, wherein said composition further comprises a fatty acid amide hydrolase inhibitor.

26. The method of claim 21, wherein said composition further comprises a derivative of OEA.

27. A method, comprising:
a) providing a oleic acid composition; and
b) treating said oleic acid composition under conditions such that an oleylethanolamide composition is generated, said oleylethanolamide composition suitable for consumption by a human or animal.

28. The method of claim 27, wherein said under conditions such that comprise reacting said oleic acid composition with a chloride to produce the acid chloride of oleic acid, reacting said acid chloride with ethanolamideaceteate to generate an acetate of oleylethanoloamide and hydrolyzing said acetate to generate oleylethanolamide.

29. The method of claim 27, further comprising the step of combining said oleylethanolamide with a food product.

30. The method of claim 27, wherein said oleic acid composition further comprises a fatty acid amide hydrolase inhibitor.

31. The method of claim 27, wherein said oleic acid composition further comprises a derivative of OEA.

32. A composition for a human or animal diet comprising a food product and an fatty acid amide component.

33. The composition of claim 32, wherein said composition is for human consumption.

34. The composition of claim 32, wherein said composition is for non-human animal consumption.

35. The composition of claim 32, wherein said composition comprises between 0.01 gram and 10.0 grams of said fatty acid amide.

36. The composition of claim 32, wherein said composition comprises between 0.5 grams and 2.0 grams of said fatty acid amide.

37. The composition of claim 32, wherein said composition comprises approximately 1.0 gram of said fatty acid amide.

38. The composition of claim 32, wherein said food product is selected from the group consisting of a beverage, a prepared food, a food ingredient, and a dairy product.

39. The composition of claim 32, wherein said fatty acid is an essential fatty acid.

40. The composition of claim 32, wherein said fatty acid is a C: 18 or longer fatty acid.

41. The composition of claim 32, wherein said fatty acid is an unsaturated fatty acid.

42. The composition of claim 32, wherein said fatty acid is a saturated fatty acid.

43. The composition of claim 32, wherein said fatty acid is a conjugated fatty acid.

44. The composition of claim 32, wherein said fatty acid is selected from the group consisting of conjugated linoleic acid, oleic acid, docosahexaenoic acid, gamma-linolenic acid, alpha linoleic acid, stearidonic acid, dihomo-gamma-linolenic acid, arachidonic acid, eicosapentaenoic acid, adrenic acid, docosahexaenoic acid, and docosapentaenoic acid.

45. The composition of claim 32, wherein said composition further comprises a fatty acid amide hydrolase inhibitor.

46. The composition of claim 32, wherein said composition further comprises an OEA derivative.
